(19) United States
(12) Patent Application Publication
(10) Pub. No.: US 2009/0024068 A1
(43) Pub. Date: Jan. 22, 2009

(54) Absorbable tissue dressing assemblies, systems, and methods formed from a Hydrophilic chitosan sponge structure and including a flexible absorbable backing layer of poly-4-hydroxy butyrate

(75) Inventors: Simon McCarthy, Portland, OR (US); Barbara McGrath, Portland, OR (US); Ervelyn Winata, Beaverton, OR (US)

Correspondence Address: RYAN KROMHOLZ & MANION, S.C., POST OFFICE BOX 26618, MILWAUKEE, WI, 53226, US

(73) Assignee: HemCon Medical Technologies, Inc.

(21) Appl. No.: 12/218,571

(22) Filed: Jul. 16, 2008

Related U.S. Application Data
(60) Provisional application No. 60/959,641, filed on Jul. 16, 2007.

(51) Int. Cl. A61F 13/02

(52) U.S. Cl. 602/43

(57) ABSTRACT

Absorbable tissue dressing assemblies are formed from hydrophilic polymer sponge structures, such as chitosan.

### RELATED APPLICATION

This application claims the benefit of U.S. Provisional Patent Application Ser. No. 60/959,641, filed Jul. 16, 2007 and entitled “Absorbable Tissue Dressing Assemblies, Systems, and Methods Formed From Hydrophilic Polymer Sponge Structures Such as Chitosan.”

### FIELD OF THE INVENTION

The invention is generally directed to tissue dressings applied on a site of tissue injury, or tissue trauma, or tissue access to ameliorate bleeding, fluid seepage or weeping, or other forms of fluid loss, as well as provide a protective covering over the site.

### BACKGROUND OF THE INVENTION

HemCon® Bandages made and sold by HemCon Medical Technologies Inc. (Portland, Ore.) incorporate a chitosan sponge matrix having superior adhesive properties and resistance to dissolution in high blood flow, which make them well suited for stanching of severe arterial blood flow.

There remains a need for improved absorbable hemostatic dressings for temporary internal use (e.g. by application by open or non-invasive procedures, such as laparoscopic procedures) or for implantation, which couple flexibility and ease of use with robustness and longevity required for resisting dissolution during use.

### SUMMARY OF THE INVENTION

The invention provides absorbable, supple, and densified tissue dressing assemblies, systems and methods formed from hydrophilic polymer sponge structures, such a chitosan. The absorbable tissue dressing assemblies make possible rapid bleeding control (usually within 1 to 2 minutes) in internal bleeding situations, such as during surgery or as the result of trauma, without using thrombin or other hemostatic agents. The tissue dressing assemblies also provide antibacterial/antiviral protection; biocompatibility; and bioabsorbability. The absorbable tissue dressing assemblies are flexible and conformable to tissue surfaces, and present a low/thin profile (e.g., less than 1 mm). They are able to be cut to a desirable size at the instant of use, and (if desired) they can be removed without tissue injury or re-bleeding by saline soaking. The absorbable tissue dressing assemblies make possible the laparoscopic delivery of hemostatic intervention to control internal bleeding episodes during surgery or as a result of trauma.

One aspect of the invention provides an absorbable tissue dressing assembly comprising an absorbable tissue dressing assembly comprising a tissue dressing matrix comprising an absorbable hydrophilic material made from chitosan, and a flexible absorbable polymer film backing layer consisting essentially of a poly-4-hydroxy butyrate biomaterial. The flexible absorbable polymer film backing layer can comprise, e.g., a mesh.

Another aspect of the invention provides a method comprising providing an absorbable tissue dressing assembly as defined above, and performing laparoscopic partial nephrectomy (LPN) using the absorbable tissue dressing assembly.

Other features and advantages of the invention shall be apparent based upon the accompanying description, drawings, and claims.

### DESCRITION OF THE DRAWINGS

FIG. 1 is a perspective exploded view of a formed hydrophilic sponge material desirably comprising a chitosan matrix, which is sized and figured as a supple, densified tissue dressing assembly.

FIG. 2 is a perspective assembled view of the supple, densified tissue dressing assembly shown in FIG. 1.

FIG. 3 is a perspective view of a sealed pouch into which the supple, densified tissue dressing assembly shown in FIG. 2 is placed and sterilized prior to use by a caregiver.

FIG. 4 is a perspective view of the pouch shown in FIG. 3 being opened by a caregiver to gain access to the supple, densified tissue dressing assembly for use.

FIG. 5 is a perspective view of the supple, densified tissue dressing assembly formed in different shapes and sizes.

FIG. 6 is a perspective view of the supple, densified tissue dressing assembly being cut to size at the instant of use.

FIG. 7 is a perspective view illustrating how the supple, densified tissue dressing assembly can be flexed, bent, folded, twisted, and even rolled upon itself before and during use, without creasing, cracking, fracturing, otherwise compromising the integrity and mechanical and/or therapeutic characteristics of the matrix.

FIG. 8 is a perspective view illustrating how the supply, densified tissue dressing assembly can be wrapped a tissue site, e.g., about a vessel to seal an anastomosis, without collapsing the vessel on itself.

FIG. 9 is a perspective view of an embodiment of a supple, densified tissue dressing assembly having a bioadsorbable backing layer sized to extend beyond the perimeter of the assembly to present a skirt of material to receive suture material or staples, to stabilize the tissue dressing assembly at the application site.

FIGS. 10 and 11 are perspective view of another embodiment of a supple, densified tissue dressing assembly having a bio-absorbable backing placed, at least in part, internally within the assembly, extending beyond the periphery of the assembly (as shown in FIG. 10) to receive suture material or staples, or be placed within the confines of the assembly (as shown in FIG. 11), through which the suture material or staples are passed.

FIGS. 12A and 12B are perspective views of another embodiment of a supple, densified tissue dressing assembly having a strip of biomaterial placed at one end of the assembly (with no such biomaterial at the opposite end of the assembly), which can be used when it is desirable to wrap the tissue dressing assembly in multiple layers around a site, e.g., to seal anastomosis from bleeding (as FIG. 12B shows).

### DETAILED DESCRIPTION

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention.

### I. Absorbable Tissue Dressing Assembly

A. Overview

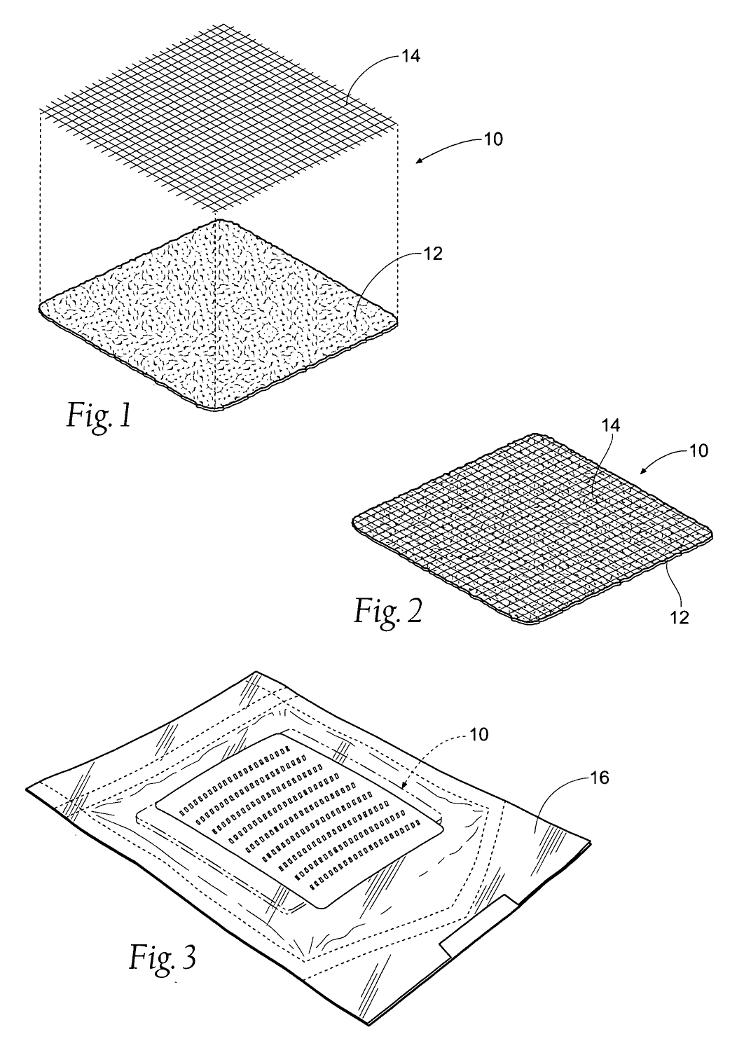

FIGS. 1 and 2 show a representative embodiment of an absorbable tissue dressing assembly 10 that embodies features of the invention. By “absorbable,” it is meant that the assembly 10 is bioabsorbable and/or biodegradable and will, in time after placement within body tissue, be broken down and/or resorbed by the body's natural processes. As shown, the absorbable tissue dressing assembly 10 comprises a relatively thin and supple tissue dressing matrix 12 (shown FIG. 1) comprising a hydrophilic polymer that can be characterized as a supple sponge structure. The absorbable tissue dressing assembly also includes a backing layer 14, which overlays one surface of the tissue dressing matrix 12. Desirably, the tissue dressing matrix 12 and the backing 14 possess different colors (e.g., a standard absorbable dressing indicating violet dye), textures, or are otherwise visually and/or tactilely differentiated, to facilitate recognition by a caregiver.

The absorbable tissue dressing assembly 10 is sized and configured as an adherent, hemostatic dressing for the rapid control of internal bleeding during surgery, in particular when ligature or conventional procedures are ineffective or impractical. The tissue dressing assembly 10 comprises a low profile, highly conformable, adherent sheet, which allows control of local and/or diffuse internal bleeding without the need for clamping or bulky packing. The size, configuration, and mechanical and physical properties of the absorbable tissue dressing assembly 10 make possible the laparoscopic delivery of hemostatic intervention to control internal bleeding episodes during surgery or as a result of trauma, e.g., during laproscopic partial nephrectomy (LPN).

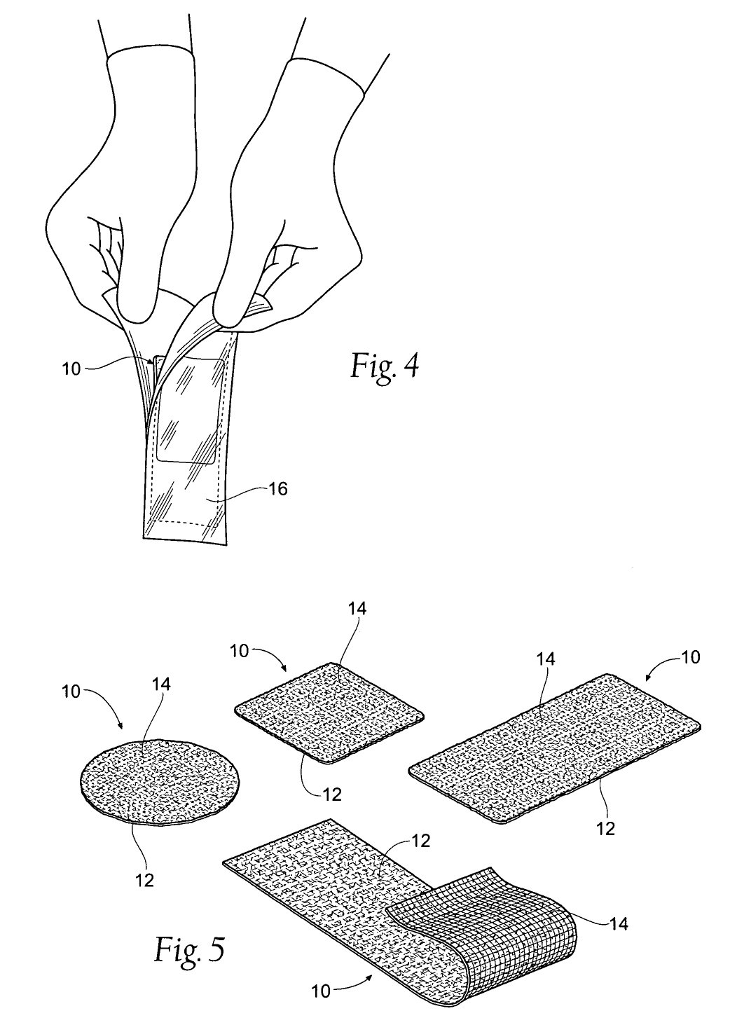

As FIG. 3 shows, the tissue dressing assembly 10 is desirably vacuum packaged in an air-tight heat sealed foil-lined pouch 16. The tissue dressing assembly 10 is subsequently terminally sterilized within the pouch 16 by use of gamma irradiation. As FIG. 4 shows, the pouch 16 is configured to be peeled opened by the caregiver at the instant of use.

B. The Tissue Dressing Matrix

The tissue dressing matrix 12 comprises a non-mammalian material poly [β-(1→4)-2-amino-2-deoxy-D-glucopyranose, which is more commonly referred to as chitosan. The chitosan can be processed in conventional ways from chitin obtained from animal crustacean shells, for example, shrimp. Chitosan is biocompatible and is biodegradable within the body, being broken down into glucosamine, a benign material.

The chitosan matrix 12 presents a robust, permeable, high specific, positively charged surface. The positively charged surface creates a highly reactive surface for red blood cell and platelet interaction. Red blood cell membranes are negatively charged, and they are attracted to the chitosan matrix 12. The cellular membranes fuse to chitosan matrix 12 upon contact. A clot can be formed very quickly, circumventing immediate need for clotting proteins that are normally required for hemostasis. For this reason, the chitosan matrix 12 is effective for both normal as well as anti-coagulated individuals, and as well as persons having a coagulation disorder like hemophilia. The chitosan matrix 12 also binds bacteria, endotoxins, and microbes, and can kill bacteria, microbes, and/or viral agents on contact. The chitosan matrix 12 is biocompatible. The chitosan matrix 12 is biodegradable within the body and is broken down into glucosamine, a benign substance, in about 4 to 6 months.

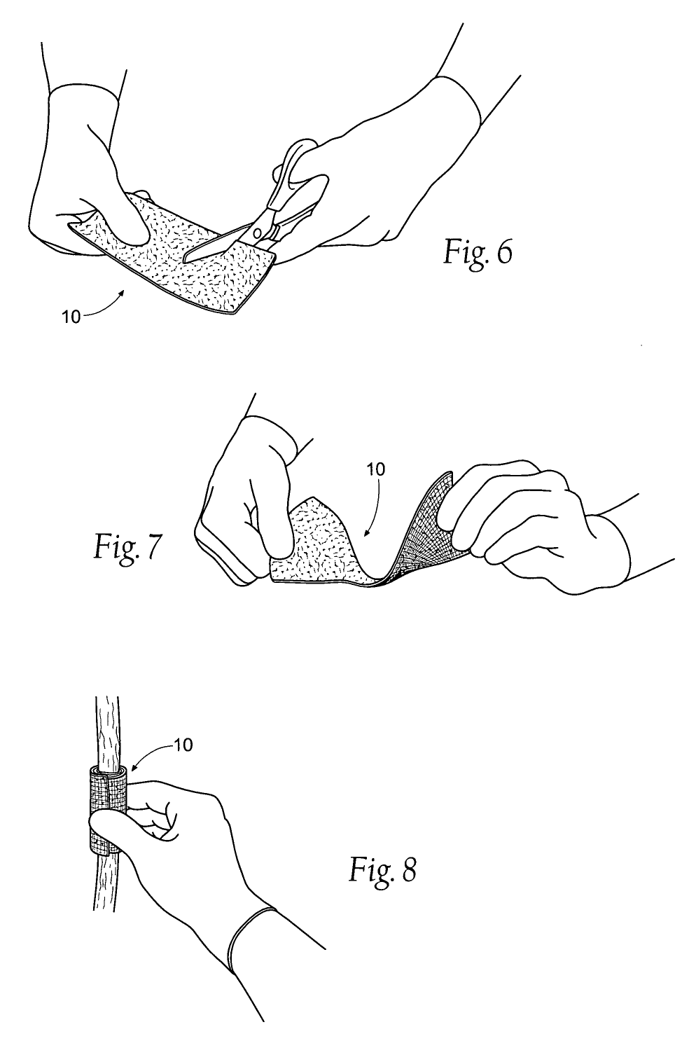

The particular size, shape, and configuration of the tissue dressing matrix 12 can, of course, vary according to its intended use. As will be described in greater detail later, the tissue dressing matrix 12 can be pre-shaped during manufacture. As FIG. 5 shows, the tissue dressing matrix can be formed in various sizes, for example, in round patches 2 to 3 inches in diameter, or in rectilinear patches, e.g., 4 inch by 4 inch, or 2 inch by 2 inch, or 2 inch by 4 inch; or as an elongated strip, which can be cut to size at the instant of use, as FIG. 6 shows.

The thickness of the tissue dressing matrix 12 can also be controlled during manufacture, as will be described in greater detail later. Desirably, the tissue dressing assembly 10 includes a thin and inherently supple chitosan matrix 12 (e.g., between 1.0 mm to 2.0 mm). The suppleness lends compliance and multi-dimensional flexibility. As FIG. 7 shows, the matrix 12 can be flexed, bent, folded, twisted, and even rolled upon itself before and during use, without creasing, cracking, fracturing, otherwise compromising the integrity and mechanical and/or therapeutic characteristics of the matrix 12.

The thin, compliant nature of the matrix 12 provides for optimal ease of application in terms of dressing flexibility and also for optimal likelihood of success in terms of attaching a strong, compliant, hemostatic matrix on or around the application site. The thin, compliant nature of the matrix 12 also makes it possible to wrap the dressing 10 about a site (see FIG. 8) (e.g., about a vessel to seal an anastomosis, or repairing accidental damage to the inferior vena cava during surgery), without collapsing the vessel on itself. A thin, compliant chitosan matrix 12, when dry prior to use, will become rapidly even more compliant when wetted with blood allowing for minimal loading on the vessel or application site during application.

C. The Backing Layer

The absorbable tissue dressing assembly 10 includes a flexible absorbable polymer film or mesh backing layer 14, which is attached to one side of the chitosan matrix 12, e.g., by melt adherence or solution casting. The flexible polymer film or mesh backing layer 14 can be attached or bonded by direct adhesion with a top layer of chitosan matrix 12. Alternatively, an adhesive such as 3M 9942 Acrylate Skin Adhesive, or fibrin glue, or cyanoacrylate glue can he employed.

The flexible polymer film or mesh backing layer 14 provides enhanced mechanical strength and impermeablity to leakage. The flexible polymer film or mesh backing layer 14 also provides an anti-adhesion barrier and a non-adherence outside surface.

The flexible polymer mesh backing layer 14 comprises poly-4-hydroxy butyrate biomaterial, commercially available as TephaFlex™ Material manufactured by Tepha Inc. In this arrangement, the poly-4-hydroxy butyrate biomaterial comprises a strong, pliable thermoplastic mesh material with a tensile strength of 50 MPa, tensile modulus of 70 MPa, elongation to break of ˜1000%, and hardness (Shore D) of 52.8. The poly-4-hydroxy butyrate biomaterial is biocompatible. In vivo, the biomaterial is hydrolyzed to 4-hydroxybutyrate, a natural human metabolite, present normally in the brain, heart, lung, liver, kidney, and muscle. This metabolite has a half-life of just 35 minutes, and is rapidly eliminated from the body (via the Krebs cycle) primarily as expired carbon dioxide.

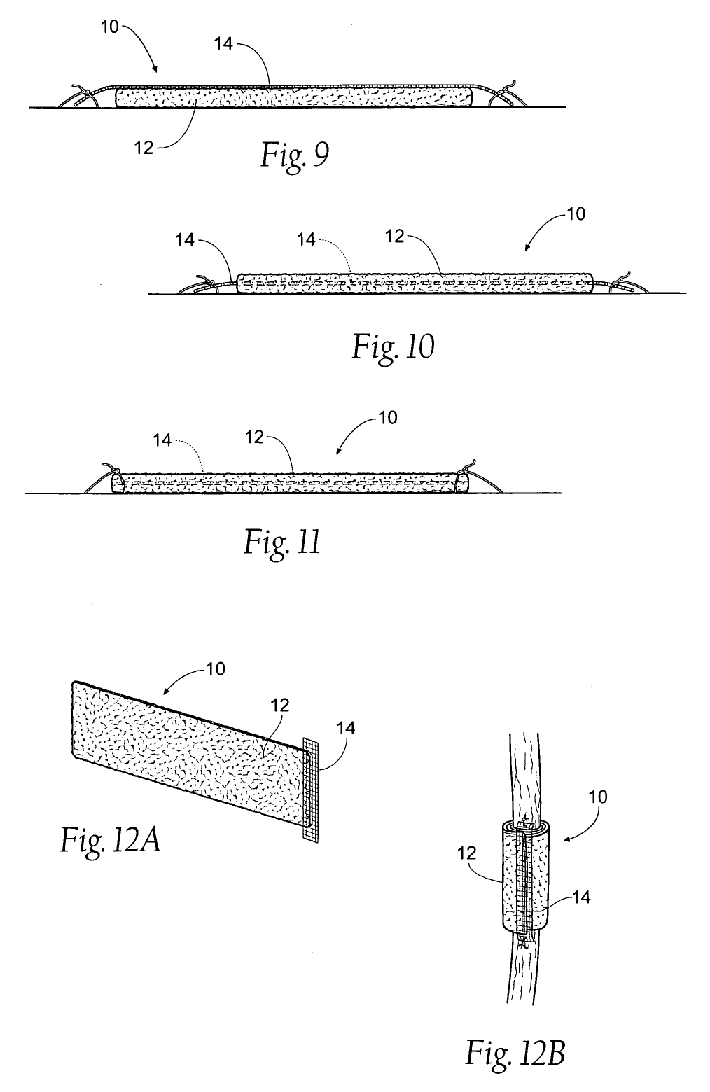

As FIG. 9 shows, the flexible bio-adsorbable backing layer 14 can be sized to extend beyond the perimeter of the chitosan matrix 12. In this arrangement, the backing layer 14 presents a skirt of material to receive suture material or staples, to stabilize the tissue dressing assembly 10 at the application site. Alternatively, the suture material or staples can be inserted through the chitosan matrix 12 itself.

As FIGS. 10 and 11 show, the biomaterial of the backing 14 can be placed, at least in part, internally within the chitosan matrix 12. The material can, like FIG. 9, extend beyond the periphery of the chitosan matrix 12 (as shown in FIG. 10) to receive suture material or staples, or be placed within the confines of the chitosan matrix 12 (FIG. 11), through which the suture material or staples are passed.

As shown in FIG. 12A, in an alternative embodiment, a strip of the biomaterial of the backing layer can be placed at one end of a thin, elongated chitosan matrix 12, with no such biomaterial at the opposite end of the matrix 12. This arrangement can be used when it is desirable to wrap the tissue dressing assembly 10 in layers around a site, e.g., to seal anastomosis from bleeding or to repair accidental damage to the inferior vena cava during surgery. In this arrangement, the length of the dressing could be designed for use on large vessels with cutting to size of the dressing by a surgeon. The chitosan matrix 12 adheres to itself when wetted with blood and allowing for at least multiple thin layers of encirclement of the anastomosis or vascular repair site by the dressing assembly 10, as FIG. 12B shows.

It should also be appreciated that, for certain indications, an absorbable tissue dressing assembly 10 may be provided without a backing layer. The absence of a backing layer may be advantageous in certain situations, e.g., when stuffing the dressing assembly into a small depressed wound, or when approximating two surface together with a layer of adhesive chitosan between them.

D. Representative Indication

The size, configuration, and mechanical and physical properties of the absorbable tissue dressing assembly 10 make possible the laparoscopic delivery of hemostatic intervention to control internal bleeding episodes during laproscopic partial nephrectomy (LPN).

### II. Manufacture of the Absorbable Tissue Dressing Assembly

The chitosan can be manufactured in the manner described in Hopman et al, US Publication No. 2007/0066920, filed Sep. 14, 2006, and entitled “Supple Tissue Dressing Assemblies, Systems, and Methods, formed from Hydrophilic Polymer Sponge Structures Such as Chitosan,” which is incorporated herein by reference.

### EXAMPLE 1

### Sealing Renal Parenchymal Wound Following Laproscopic Partial Nephrectomy

Nephron sparing surgery (NSS) has become a standard treatment in patients with a compromised contralateral kidney or in select patients with a favorably located, small renal tumor with a normal contralateral kidney.

While minimally invasive surgery is becoming established for radical nephrectomy, similar approach for nephron sparing surgery have been limited largely due to technical issues such as achieving adequate renal parenchymal hemostasis, and secure caliceal closure. Due to these technical difficulties associated with minimally invasive approach the current complication rates related to urinary leakage and hemorrhage following laparoscopic partial nephrectomy (LPN) remains significantly higher than the open NSS.

At present time, LPN remains technically challenging due to, lack of a reliable method for obtaining consistent parenchymal hemostasis, and the technical difficulties in obtaining secure suture closure of the renal collecting system. Various topical sealants including fibrin glue, gelatin resorcinol formaldehyde glue, and oxidized cellulose or gelfoam sponges are frequently used alone or in conjunction with specialized instrumentation and agents to assist hemostasis. However, these topical preparations mostly require renal hilar vascular control during the application and are unable to seal urinary collecting system effectively. Moreover, the currently topical preparations are mostly composed of heterogenic protein, which may bear a potential risk of severe allergic reaction and possible viral contamination.

An absorbable tissue dressing assembly 10 comprising a chitosan matrix 12 frozen, freeze dried, densified, and softened, as described herein) with a poly-4-hydroxy butyrate backing layer 14 (TephaFlex™ Material manufactured by Tepha Inc) (the “chitosan hemostatic dressing”) was evaluated for renal parenchymal wound sealing to control hemorrhage and urine leakage in laparoscopic partial nephrectomy (LPN). The chitosan hemostatic dressings used were 58 mm in diameter and between 1.5 mm to 1.85 mm thick with a density, after densification, of between 0.12 g/cm³ and 0.15 g/cm³. The poly-4-hydroxy butyrate backing layer 14 was 25-μm thick. The backing side faces up on application and the other side, or “active side”, is the side directly applied to the wound surface.

Forty-five LPN were performed on twenty-four domestic swine. After excision of the lower pole of the kidney, either the chitosan hemostatic dressing (N=24) was applied to seal the wound, or conventional suture technique (N=21) (the control group) was used to control bleeding. The operative parameters included estimated blood loss (EBL) and operative time (OT) were recorded. The animals were euthanized at 1 hour, 3 days and 7 days postoperatively. Retrograde ureteropyelography was performed to assess the urine leakage.

All animals achieved initial hemostasis and survived with both treatments. Mean EBL was significantly lower when the chitosan hemostatic dressing was used, when compared to it in the control group (128 ml EBL for the chitosan hemostatic dressing vs. 363 ml EBL for the control group, p=0.04).

There was no significant difference in OT between the two treatments.

In the control group, all kidneys present urine leakage after LPN in both acute and chronic periods. In the chitosan group, 8% ( 1/12) of kidneys had acute urine leakage, 33% of them developed small urinoma at postoperative day 3 and 7, respectively. Histopathological analysis showed similar and mild informatory responses in both groups.

The chitosan-based hemostatic dressing proved effective as a primary or supplemental treatment for sealing the parenchymal wound in laparoscopic partial nephrectomy in the animal model.

### EXAMPLE 2

### Use of a Chitosan-Based Hemostatic Dressing in Laproscopic Partial Nephrectomy

An absorbable tissue dressing assembly 10 comprising a chitosan matrix 12 frozen, freeze dried, densified, and softened, as described herein) with a poly-4-hydroxy butyrate backing layer 14 (TephaFlex™ Material manufactured by Tepha Inc) (the “chitosan hemostatic dressing”) was evaluated for renal parenchymal wound sealing to control hemorrhage and urine leakage by sealing off the renal parenchymal wound surface in LPN procedures.

The chitosan hemostatic dressings used were 58 mm in diameter and between 1.5 mm to 1.85 mm thick with a density, after densification, of between 0.12 g/cm³ and 0.15 g/cm³. The poly-4-hydroxy butyrate backing layer 14 was 50-μm thick. The backing side faces up on application and the other side, or “active side”, is the side directly applied to the wound surface.

Nine heparinized domestic swine underwent bilateral laparoscopic partial nephrectomies involving either a polar (N=13) or wedge resection (N=5) followed by treatment with the chitosan hemostatic dressing. Estimated blood loss, hemostatic score, urinary leakage, operative time and adhesion score of the chitosan dressing were recorded.

After induction of general anesthesia, all the procedures were performed under general anesthesia with intubation using strict aseptic precautions. The animal was placed in a lateral position and CO2 pneumoperitoneum was created using a 14-gauge Veress needle. Two 10 mm and two 5 mm ports were placed. A bolus of 5000 units of intravenous heparin was given 10 minutes prior to operation and additional bolus dosages of 1000 units were given intraoperatively every 30 minutes as required in order to maintain the activated clotting time (ACT) over 200 seconds and rechecked throughout the surgical procedure. ACT was rechecked every 30 minutes. If ACT dropped below 200 additional 1000 units of heparin were re-given until it was above 200.

The kidney was identified and following exposure of renal hilum both the poles were completely mobilized. Either an upper or a lower polar, or a wedge resection was performed using a harmonic scalpel (Ethicon Endosurgery, Cincinnati, Ohio) without hilar occlusion. In the polar resection, at least one third of renal tissue was resected; in the wedge resection, an approximate 3 cm in depth and 3 cm in width tissue was removed from middle of kidney, which making sure the collecting system was entered by visual confirmation. The hemorrhage through the parenchymal surface of the kidney was assessed visually by assigning a 0-4 hemostatic score (0=no hemostasis; 1=steady bleeding; 2=moderate bleeding; 3=mild oozing; and 4=dry).

An appropriate sized piece of chitosan dressing was delivered through a 10-mm port and deployed onto the resected surface for 3 minutes with gentle compression using a 10-mm fan retractor (Endo Retract, Auto Suture□, U.S Surgical, Norwalk, Conn.). Hemostatic score was recorded again. The ability of the dressing to adhere securely to the entire cut surface of the renal parenchyma was assessed on a numeric scale of 0-5 (0=no adhesive; 1=weakly adhering to one small portion of surface; 2=uniformly but weakly adhering to whole surface; 3=general weak with some moderate adherence; 4=limited but strong local adherence; and 5=uniform strong adherence). In case of non or incomplete adherence of the dressing to the renal parenchyma the dressing was removed and a second piece was deployed. The numbers of attempts required for successful deployment were recorded. After completion of satisfactory hemostasis and secure adhesion of the dressing on one side, the animal was turned onto the contralateral side and the procedure was repeated.

Once both sides had achieved initial hemostasis, the abdomen was deflated and additional 30 minutes were given to observe the stability of the repairs. Pneumoperitoneum was reestablished and the repair sites were reassessed laparoscopically for any evidence of rebleeding or urinary extravasation and to obtain the final hemostatic score. A retrograde pyelography was performed to assess the integrity of the collecting system and pyelocaliceal urinary leakage via bilateral ureteral catheterization under cystotomy. Finally the animals were euthanized and both kidneys were removed through midline laparotomy for gross assessment of the quality of adhesion of the dressing. A thumbprint of the resected portion of the kidney was obtained on a graph paper to determine the area of resection.

Of 18 procedures, 17 achieved complete hemostasis after deployment of the chitosan hemostatic dressing. The hemostasis score improved significantly after the deployment in both polar (p<0.001) and wedge (p=0.017) resections. The rate of successful pyelocaliceal sealing was 85% ( 11/13) in polar and 60% (⅗) in wedge resections.

The chitosan hemostatic dressing is effective as a primary or supplemental material for controlling parenchymal hemorrhage and sealing the renal collecting system following LPN in the animal model.

Due to characteristic physical properties of chitosan hemostatic dressing, secure application of this material is feasible and technically less challenging following LPN. For the best result it is necessary to hold the dressing firmly and steady against the wound surface of renal resection for approximately 3 minutes during the operation.

In the case of polar resection, this goal is easily accomplished with commercially available fan-type retractor. All of the polar resections achieved complete hemostasis with this technique (100%, 13/13) within a reasonably short duration of time.

Closure of pyelocalceal system traditionally requires watertight suture closure. Laparoscopically this task is technically challenging and time consuming. The ability of the chitosan hemostatic dressing to adhere strongly to the freshly incised raw surface of the solid organs provides a unique opportunity to seal the pyelocalceal system. The chitosan hemostatic dressing was able to seal the pyelocalceal system securely without any evidence of urinary leakage on follow-up retrograde pyelography in majority (85%, 11/13) of the polar resections. The success rate was about the same or better than that reported with traditional techniques.

In the case of wedge resections, the amount of blood loss and the number of attempts required to apply the dressing were relatively greater. Also the quality of adhesion of the chitosan hemostatic dressing to the freshly incised renal parenchyma was poorer leading to worse hemostatic scores and higher rate of urinary leakage (⅖, 40%). Typically, application of the chitosan hyemostatic dressing via laparoscopic approach in wedge resections is more technically challenging due to the awkward V-shape configuration of the renal parenchymal injury and a lack of appropriate instruments that would allow maintenance of firm and uniform firm pressure over the dressing for three minutes (the minimum desired time for achieving secure adhesion of the dressing). Further developments are required to improve instrumentation for the deployment of the chitosan dressing in laparoscopic wedge resections.

The chitosan hemostatic dressing provides an easy and rapid method to control bleeding and seal the parenchymal wound surface. Use of the chitosan dressing can simplify LPN procedure to save operative time, and it can be used without hilar vascular occlusion to avoid renal warm ischemia. Clinical situations for possible use of the chitosan dressing in laparoscopic surgery are numerous. Bleeding from visceral organs after routine biopsy and resection, portal bleeding, or more severe bleeding from hilar vascular injury, and surgical bleeding from coagulopathy patients can be controlled using the chitosan hemostatic dressing as well.

This example presents promising results for achieving immediate hemostasis and sealing urinary leakage with the use of the chitosan hemostatic dressing following laparoscopic polar or wedge resection of the kidney in a porcine model. The technique is technically less demanding and allows rapid control of hemorrhage and sealing of severed pyelocaliceal system. The technique also has several potential applications including laparoscopic control of hemorrhage form solid organs as a result of surgical injury or following trauma.

### CONCLUSION

It should be apparent that above-described embodiments of this invention are merely descriptive of its principles and are not to be limited.

We claim:

1. An absorbable tissue dressing assembly comprising
a tissue dressing matrix comprising an absorbable hydrophilic material made from chitosan, and
a flexible absorbable polymer film backing layer consisting essentially of a poly-4-hydroxy butyrate biomaterial.

2. An assembly according to claim 1
wherein the flexible absorbable polymer film backing layer comprises a mesh.

3. A method comprising
providing an absorbable tissue dressing assembly as defined in claim 1 or 2, and
performing laparoscopic partial nephrectomy (LPN) using the absorbable tissue dressing assembly.
